(19) United States
(12) Patent Application Publication
(10) Pub. No.: US 2004/0186084 A1
(43) Pub. Date: Sep. 23, 2004

(54) Triamcinolone formulations and methods for their preparation and use

(75) Inventors: Abu Alam, Lake Forest, IL (US); Hari Menon, Bellemead, NJ (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Akorn, Inc., Buffalo Grove, IL

(21) Appl. No.: 10/394,821

(22) Filed: Mar. 21, 2003

(51) Int. Cl. A61K 31/58

(52) U.S. Cl. 514/174

(57) ABSTRACT

Sterile compositions comprising triamcinolone compounds (desirably, triamcinolone acetonide), systems for providing such compositions, and methods for the preparation and use of such compositions. Exemplary of the inventive compositions is a sterile aqueous composition comprising triamcinolone acetonide, wherein the sterile aqueous composition has a pH of between about 5 and 7.5 and contains no more than 1 wt. % excipients other than tonicity-adjusting agents and pH-adjusting agents.

### FIELD OF THE INVENTION

This invention pertains to injectable formulations comprising pharmaceutically-acceptable compounds of triamcinolone and methods for their preparation and use.

### BACKGROUND OF THE INVENTION

Triamcinolone compounds, and specifically the acetonide ((11β,16α)-9-fluoro-11,21-dihydroxy-16,17-[1-methylethylidenebis(oxy)]pregna-1,-4-diene-3,20-dione), are categorized as a glucocorticoids (synthetic corticosteriods). The acetonide compound is typically provided in crystalline form (mp 292-294° C.). While triamcinolone acetonide is relatively insoluble in water, it is sparingly soluble in methanol, acetone and ethyle acetate.

Generally useful as an antiasthmatic, antiallergenic and anti-inflammatory, triancinolone acetonide has been included as the active ingredient in many pharmaceutical formulations, such as creams, ointments, and tablets.

Triamcinolone acetonide has also been included as the active pharmaceutical ingredient (API) in injectable formulations. Examples of such injectable formulations include Kenalog® (Apothecon) and TAC™ (formerly marketed by Allergan, Inc.). Kenalog® is available in two strengths: 10 mg/ml (Kenalog®-10) and 40 mg/ml (Kenalog®-40). The former is suitable for intradermal, intra-articular and intrabursal injection, while the latter is suitable for intramuscular and intra-articular injection. Allergan, Inc. has in the past marketed TAC™-3 and TAC™-40 in 3 mg/ml and 40 mg/ml strengths, respectively.

In addition to the active ingredient triamcinolone acetonide, the Kenalog® formulations contain a number of excipients (inactive ingredients). More specifically, and in addition to water, each ml of Kenalog® (10 mg/ml or 40 mg/ml formulation) includes the following excipients: sodium chloride for isotonicity, 0.9% (w/v) benzyl alcohol as a preservative, 0.75% sodium carboxymethylcellulose, and 0.04% polysorbate 80, with sodium hydroxide or hydrochloric acid possibly being present to adjust the pH to between 5.0 and 7.5.

While the foregoing triamcinolone acetonide injectable formulations have been used for years in the successful treatment of patients, a need exists for formulations that provide certain advantage over existing formulations. The specifics regarding the present invention, and the advantages flowing therefrom, are described in detail herein.

### BRIEF SUMMARY OF THE INVENTION

The present invention provides, in one aspect, an aqueous sterile triamcinolone acetonide composition consisting essentially of triamcinolone acetonide, water, a tonicity-adjusting agent in an amount sufficient to provide the sterile aqueous composition with a physiologically-acceptable tonicity, and a pH-adjusting agent in an amount sufficient to provide the sterile aqueous composition with a pH of from about 5.0 to about 7.5.

In a related aspect, the present invention provides a sterile aqueous composition comprising triamcinolone acetonide, wherein the sterile aqueous composition has a pH of between about 5.0 and 7.5 and contains no more than 1 wt. % excipients other than tonicity-adjusting agents and pH-adjusting agents.

Further aspects of the invention contemplate providing unit doses of the foregoing sterile aqueous triamcinolone acetonide compositions, and a system for providing such sterile unit doses. The latter contemplates, in one aspect, providing a unit dose of sterile triamcinolone acetonide in a first container, and a second container comprising a sterile composition of water and a tonicity-adjusting agent. The unit dose and system aspects of the present invention also contemplate, if desired, providing sterile triamcinolone acetonide, preferably in sterile unit doses, as a lyophilizate suitable for reconstitution.

Other aspects of the present invention contemplate processes for preparing the inventive compositions, as well as the use thereof in connection with any presently recognized therapy for which triamcinolone acetonide is indicated. In one preferred aspect, the inventive formulations may be used in the treatment of ocular edema, and advantageously in the treatment of macular edema, either alone or in conjunction with other therapies. In a further preferred aspect, the inventive formulations may be used in the treatment of age-related macular degeneration (ARMD), advantageously with other therapies.

### DESCRIPTION OF THE FIGURES

FIG. 1 is a HPLC chromatogram of an aqueous triamcinolone acetodine composition of the Example prior to stability testing.

FIG. 2 is a HPLC chromatogram of the same aqueous triamcinolone acetodine composition used in obtaining the chromatogram of FIG. 1 after 4 weeks storage in a stoppered vial at 40° C. and ambient humidity.

FIG. 3 is a particle size distribution of the same aqueous triamcinolone acetodine composition used in obtaining the chromatogram of FIG. 1 prior to stability testing.

FIG. 4 is a particle size distribution of the same aqueous triamcinolone acetodine composition used in obtaining the chromatogram of FIG. 1 after 4 weeks storage in a stoppered vial at 40° C. and ambient humidity.

### DETAILED DESCRIPTION OF THE INVENTION

In its various aspects, the present invention provides sterile compositions comprising triamcinolone salts (desirably, triamcinolone acetonide), systems for providing such compositions, and methods for the preparation and use of such compositions.

In one aspect, the present invention contemplates sterile aqueous compositions of triamcinolone acetonide which, relative to known compositions, contain reduced amounts of certain excipients (inactive ingredients). Despite this reduction in excipients, it was unexpectedly found that the desirable properties of the triamcinolone acetonide compositions were not unduly compromised. Further, it is believed that this reduction provides certain benefits relative to known triamcinolone acetonide compositions, e.g., a reduction in side effects.

One of the inventive sterile triamcinolone acetonide compositions consists essentially of triamcinolone acetonide, water, a tonicity-adjusting agent in an amount sufficient to provide the sterile aqueous composition with a physiologically-acceptable tonicity, and a pH-adjusting agent in an amount sufficient to provide the aqueous composition with a pH of from about 5.0 to about 7.5.

A related aspect of the present invention provides a sterile aqueous composition comprising triamcinolone acetonide, wherein the sterile aqueous composition has a pH of between about 5.0 and about 7.5 and contains no more than 1 wt. % excipients other than tonicity and pH-adjusting agents.

In the foregoing and other aspects of the present invention, the triamcinolone acetonide used therein is well-known to those skilled in the art, and may be prepared by any suitable method. Exemplary methods of preparing this active pharmaceutical ingredient (API) are described in U.S. Pat. Nos. 2,990,401 and 3,035,050.

While the triamcinolone acetonide may be provided in any suitable physical form, it is desirably provided in the form of microparticles (also referred to as micronized triamcinolone acetonide). These microparticles are the result of a process in which crystalline (and relatively water-insoluble) triamcinolone acetonide is milled until the API possesses certain physical properties, typically so that 95% of the particles have a size of no more than about 100 microns. Exceeding this 100 micron is undesirable as it may cause local irritation at the injection site, and may further increase the dissolution (and absorption) time, undesirably delaying the therapeutic activity of the API. Desirably, the triamcinolone acetonide may be milled so it possesses an average particle size of no more than 20 microns, preferably no more than 15 microns, and most preferably no more than 10 microns, prior to its inclusion in the inventive compositions and related methods for their preparation. It is desirable to further mill the particles so that not less than 10% of the particles are no more than 5 microns (preferably no more than 2 microns), 50% of the particles are no more than 20 microns (preferably no more than 10 microns), and/or not less than 95% of the particles are no more than 100 microns (preferably no more than 50 microns). Most preferably, the particles may possess an average particle size of about 10 microns, and 95% of the particles may have a particle size of no more than 100 microns (more preferably no more than 50 microns).

The average particle size and distribution of the triamcinolone acetonide may be determined using a Malvern MasterSizer Instrument (Malvern Instruments, Ltd., Worcestershire, United Kingdom). When triamcinolone acetonide alone is evaluated, the sample to be tested may be prepared by sonicating, for 10 seconds, a mixture of about 100 mg of the particles in about 15 ml of water. The resulting sample may then be added drop wise into the measuring cell of the instrument (previously filled with water and corrected to compensate for the use of water as the medium) until the optimum obscuration level is reached. The particle size parameters may then determined in accordance with the procedures appropriate for the instrument.

Because triamcinolone acetonide is relatively insoluble in water, the aqueous compositions of the present invention may be characterized as aqueous suspensions, particularly when the triamcinolone is provided in the form of microparticles, as described above. These suspensions have been found to be therapeutically acceptable and effective, despite the relatively low levels, or preferably the absence of, excipients such as solubilizers (co-solvents, surfactants and the like) and thickeners. The inventive compositions provide a very low level (no more than that set forth in the U.S. Pharmacopoeia, or USP) of endotoxins (endotoxins not being removed by sterilization).

The amount of triamcinolone acetonide in the inventive compositions may vary, depending on the desired use without departing from the present invention. Generally, the amount of this API in the sterile aqueous compositions may range from about 1 mg to about 80 mg, desirably from about 3 to about 40 mg/ml, and preferably at about 3 mg, about 10 mg or about 40 mg, based upon 1 ml of the sterile aqueous composition.

The inventive compositions may further contain a tonicity-adjusting agent and a pH-adjusting agent.

A wide variety of tonicity-adjusting agents are known, and may be used in amounts sufficient to impart a physiologically-acceptable tonicity to the sterile aqueous compositions. Desirably, the tonicity agent may be sodium chloride, potassium chloride, dextrose, glycerin, alanine, and mixtures thereof. Preferably, sodium chloride may be used as the tonicity-adjusting agent.

A number of different pH-adjusting agents are known, and may be used as desired to impart a physiologically-acceptable pH to the sterile aqueous compositions. Desirably, the pH-adjusting agent may be a buffer or inorganic base, and may preferably be a base such as sodium hydroxide and potassium hydroxide. The desired pH of the sterile aqueous composition prior to use may range from about 5 to about 7.5, but may preferably range from about 6 to about 7 and more preferably from about 6 to about 7, most preferably about 6.5.

As previously mentioned, the sterile aqueous compositions of the present invention desirably contain little, or preferably no, excipients other than a tonicity-adjusting agent and, optionally, a pH-adjusting agent. The level of these other excipients is desirably no more than 1 wt. %, more desirably no more than 0.5 wt. %, and preferably no more than 0.1 wt. %. More preferably, the sterile compositions are substantially free of excipients other than tonicity and pH-adjusting agents, i.e., no more than 0.01 wt. %, and most preferably they are free of such other excipients.

Illustrative excipients other than tonicity and pH-adjusting agents are preservatives, alcohols, suspending agents (e.g., surfactants), and thickening agents. In limiting these other excipients, the inventive sterile aqueous compositions (and aqueous lyophilizate reconstituting solutions) desirably contain reduced amounts (e.g., in the weight percentages set forth in the preceding paragraph) of one, of a plurality, or most preferably of all, other excipients. In this regard, the sterile aqueous compositions may have varying levels of one or more of a preservative, an alcohol, a suspending agent and a thickening agent, with the total amount of such other excipients desirably remaining below the aforementioned weight percent levels. Preferably, the sterile aqueous compositions are substantially free (and more preferably free) of at least one of, at least two of, at least three of, or at least four of, a preservative, an alcohol, a suspending agent and a thickening agent.

The foregoing sterile aqueous triamcinolone acetonide compositions may be packaged in bulk, but may preferably packaged in unit dose form. This packaging may take any suitable form, e.g., a glass or plastic vial, or pre-loaded syringe. Illustrative of this dosage form is a two-container system. In this system, one container would include a sterile composition comprising a single dose of sterile triamcinolone acetonide, preferably in micronized form or alternatively, and more preferably, as a lyophilizate. If desired, the tonicity and/or pH-adjusting agents (or other excipients) could be included with the triamcinolone. A second container would contain a sterile composition comprising, at least, water. Preferably, the second container may further contain one or more of the tonicity and/or pH-adjusting agents, as well as any other water-soluble or water-dispersible excipients, as desired.

The inventive sterile aqueous triamcinolone acetonide compositions are also stable. For example, when placed under accelerated stability testing (40° C., ambient humidity), a high pressure liquid chromatography (HPLC) assay (per U.S. Pharmacopoeia, USP) for the active determined that the active was present at no less than about 90% of label claim after 3 weeks of storage, and no less than about 80% label claim after 4 weeks of storage. Further, the level of a single degradant during one or more of the aforementioned stability test periods desirably does not vary by more than about 25%, and preferably by more than about 15%, from its initial value (i.e., the level prior to the aforementioned accelerated stability testing). On an absolute basis, for one or more of the test periods, the maximum amount of a single degradant desirably remains below about 0.30%, and preferably no more than about 0.20%, of the sterile aqueous composition. The total degradant level observed during one or more of the aforementioned stability test periods desirably does not vary more than about 30%, preferably no more than about 20%, from its initial value (i.e., the level prior to the aforementioned accelerated stability testing). On an absolute basis, for one or more of the test periods, the maximum amount of total degradants desirably remains below about 0.30%, and preferably no more than about 0.20%, of the sterile aqueous composition.

It was found that the lyophilization process provided advantages in the preparation of the inventive compositions. The preferred micronized triamcinolone acetonide is a fluffy powder that has a tendency to cake. Using lyophilization, a non-caking free-flowing powder material can be provided.

The process of lyophilization is well-known to those skilled in the art, and will not be described in detail herein. In the context of the present invention, the lyophilization process may be undertaken in bulk, but is preferably conducted in a unit dose container. The present invention desirably contemplates combining sterile triamcinolone acetonide (which is desirably in micronized form) with water (desirably sterile WFI), the tonicity and pH-adjusting agents, and thereafter subjecting the resulting composition to lyophilization. The amounts of each component included in the pre- and post-lyophilization composition would be as described herein. Thereafter, the free-flowing powdery lyophilizate may be sterilized to provide a sterile dry composition for reconstitution with sterile water.

The triamcinolone acetonide may also be provided as a liposomal composition. In this form, the release of the API within the tissue may be controlled or extended relative to compositions in which the API is used in micronized form. Processes for providing this API in liposomal form are well known to those skilled in the art, and will not be described in detail herein.

The sterilization of the components in the containers may be undertaken by any suitable means, with the triamcinolone acetonide being desirably sterilized before introduction into the container. Sterilization is preferably completed using ethylene oxide gas. Sterilization by ethylene oxide has the advantage over other sterilization methods in that the former avoids the potential introduction of impurities into the sterile composition.

Reconstitution of the sterile lyophilized triamcinolone composition (or the addition of sterile aqueous composition to triamcinolone powder) may be undertaken using a needle, whereby a measured amount of the aqueous composition (to provide the desired concentration of the API) is withdrawn from a second container and introduced into the first container containing the triamcinolone API. After mixing (e.g., shaking), the finished sterile triamcinolone acetonide composition is withdrawn from the first container, and administered to a patient to provide the desired therapy.

The triamcinolone acetonide compositions described herein may be used in any method wherein therapy with this API is desired. For example, in the treatment of: endocrine disorders (nonsuppurative thyroiditis), rheumatic disorders (arthritis, bursitis, epicondylitis, tenosynovitis, ankylosing spondylitis), collagen diseases (e.g, systemic lupus, rheumatic carditis), dermatologic diseases (pemphigus, Stevens-Johnson syndrome, dermatitis, psoriasis), allergic states (e.g., asthma, dermatitis, allergic rhinits), ophthalmic diseases (e.g., atrophic macular degeneration (AMD), neovascular degeneration (NVD), choroidal neovascularization (CNV)), chronic allergic and inflammatory processes (e.g., herpes zoster, iritis, iridocyclitis, chorioretinitis, diffuse posterior uveitis and choroiditis, optic neuritis, sympathetic ophthalmia, anterior segment inflammation), gastrointestinal disease (e.g., ulcerative colitis, regional enteritis), respiratory diseases (e.g., sarcoidosis, berylliosis, pneumonia), hematologic disorders (e.g., acquired hemolytic anemia), neoplastic diseases (e.g., leukemia, lymphoma), edematous state (e.g., to induce diuresis or remission of proteinuria in the nephritic syndrome, without uremia, of the idiopathic type or that due to lupus erythematosus), chronic pain, temporal arteritis, myasthenia gravis, and diabetic macular edema.

In treating the aforementioned conditions, the inventive compositions may be administered by intra-articular, intradermal or intramuscular injection. For example, in providing therapy to the eye, such as in the treatment of edema, such as macular edema, or CNV, an inventive composition is injected directly into the eye tissue (intravitreous) into or adjacent the edema using a fine gauge (e.g., 27 gauge) needle in an amount sufficient to provide the desired therapy. In such therapy, relatively small amounts of the composition may be injected into the eye, typically from about 0.05 ml to about 0.2 ml of a triamcinolone acetonide composition having a concentration of the API of from about 10 mg to about 40 mg per ml of the composition. Preferably, a 40 mg/ml concentration is used, providing for the administration of from about 1 mg to about 8 mg of the API to the affected tissue.

Other active pharmaceutical ingredients may be administered to a patient in conjunction with the triamcinolone acetonide therapies described herein. For example, antibiotics, anti-angiogenesis agents, and/or anti-vascular endothelial growth factor may also be injected into the affected tissue.

The therapies described herein may also be used subsequent to various diagnostic procedures. For example, angiography may be used to diagnose ocular (or macular) edema. Angiography is a well-known procedure, and may be completed using one or a number of suitable dyes including tricarbocyanine dyes. Of these dyes, indocyanine green (IC-GREEN™, Akorn, Inc.) is preferred.

Other well-known therapies for ocular conditions, e.g., treatments using the application of lasers for, among others, CNVs, may also be undertaken in conjunction with the triamcinolone acetonide therapy described herein. Of these, photodynamic therapy (PDT) and photocoagulation of the CNV or vessels that permit blood to enter the CNV (i.e., feeder vessels) may be used prior to the administration of the sterile triamcinolone acetonide compositions. If photocoagulation is undertaken, it may be completed without or, preferably with, the use of a suitable dye, such as indocyanine green. Typically, PDT uses a porphyrin (e.g., verteprofin) to effect therapy. These procedures are more fully described in U.S. Pat. Nos. 6,351,663 and 6,443,976. The inventive compositions may also be used to treat a patient afflicted with edema or bleeding following vitreal or retinal surgery via injection of a therapeutically-effective amount of the inventive sterile aqueous composition into the eye in the vicinity of the edema or bleeding.

The following example further illustrates the invention but, of course, should not be construed as in any way limiting its scope.

### EXAMPLE

This example demonstrates an illustrative sterile aqueous triamcinolone acetonide composition (suspension) of the present invention, and the stability associated therewith over time.

A composition was prepared by adding sodium hydroxide to water for injection (WFI) residing in a first container. The amount of sodium hydroxide was sufficient to provide the resulting composition with a pH of about 10. Sodium chloride (9.9 mg/ml) was then mixed into the composition until dissolved. The resulting aqueous composition was then sterilized via passage through a 0.2 micron filter.

Triamcinolone acetonide (micronized, at 40 mg/ml) was then added to the aqueous composition. A one (1) ml quantity of the resulting aqueous triamcinolone acetonide composition was then introduced into a glass vial, the latter then being stoppered. The vial was then subjected to accelerated stability at 40° C.

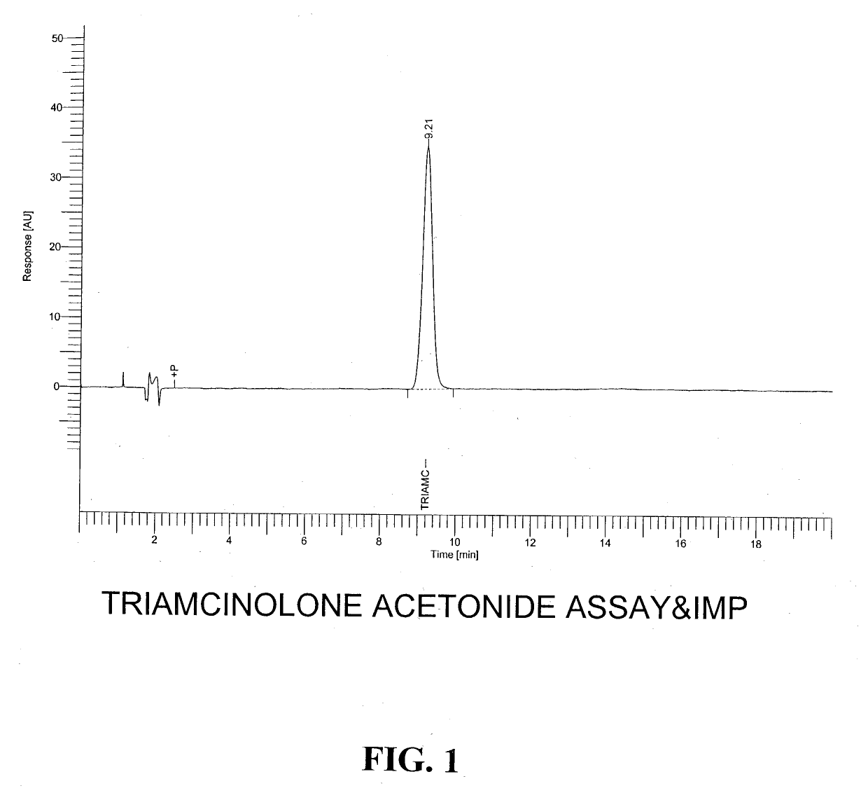

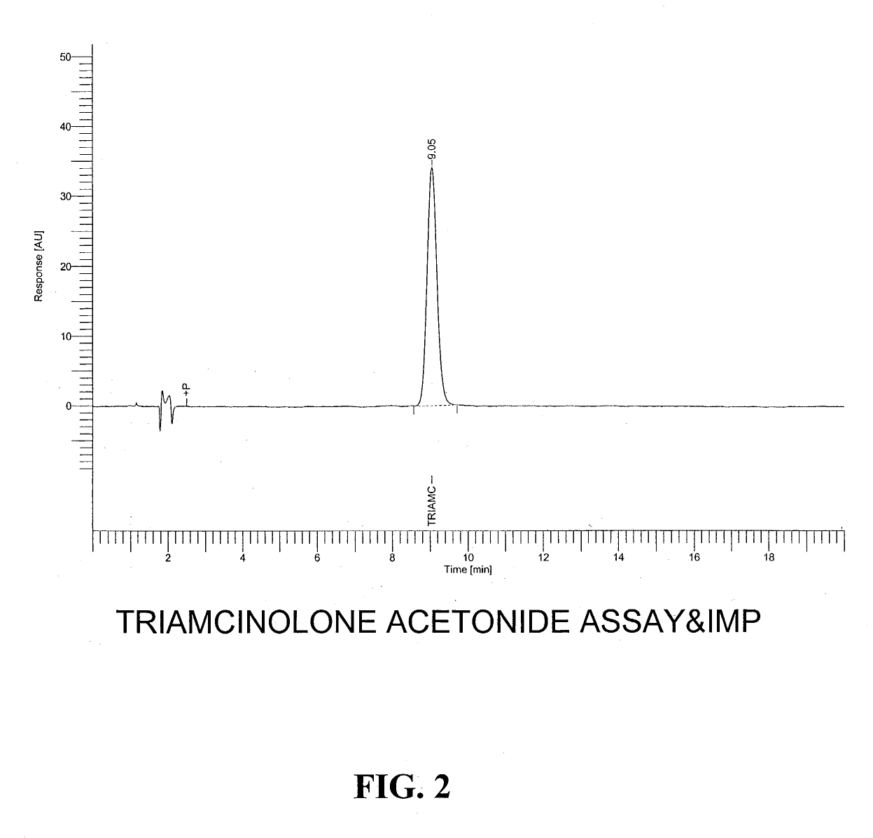

Table I shows the stability data of composition initially, and at 1, 3 and 4 weeks storage, at 41° C. (ambient humidity). Triamcinolone acetonide was assayed by the HPLC method described in the USP. The results show that there was no significant change in degradation (single peak or total) during the storage period, demonstrating good stability. FIGS. 1 and 2 are HPLC chromatograms of the API at initial and after 4 weeks storage. Moreover, there was no change in the formulation over each of the periods (1, 3 and 4 weeks) with respect to physical appearance (visible), color (visible), pH or osmolality.

TABLE 1

| | Initial | Week 1 | Week 3 | Week 4 |
|---|---|---|---|---|
| HPLC Assay | 88.5% | 97.0% | 95.5% | 79.0% |
| (Label Claim) | | | | |
| Maximum Single Degradant | 0.17% | 0.17% | 0.19% | 0.15% |
| (% of composition) | | | | |
| Total Degradant (% of | 0.18% | 0.17% | 0.21% | 0.15% |
| composition) | | | | |
| pH | 6.16 | 5.93 | NM | NM |
| Osmolality | 313 mOsm | 314 mOsm | 322 mOsm | 330 mOsm |

NM = Not measured

The particle size of the micronized triamcinolone acetonide aqueous suspension was also determined initially, and after 1, 3 and 4 weeks of storage at 40° C. The particle size was determined by transferring about 10 ml of the aqueous suspension into a beaker. The suspension was then sonicated for 10 seconds. The resulting sample was added drop wise into the measuring cell of a Malvern Master-Sizer instrument (previously filled with water and corrected to compensate for the use of water as the medium) until the optimum obscuration level is reached. The particle size parameters were then determined in accordance with the procedures appropriate for the instrument.

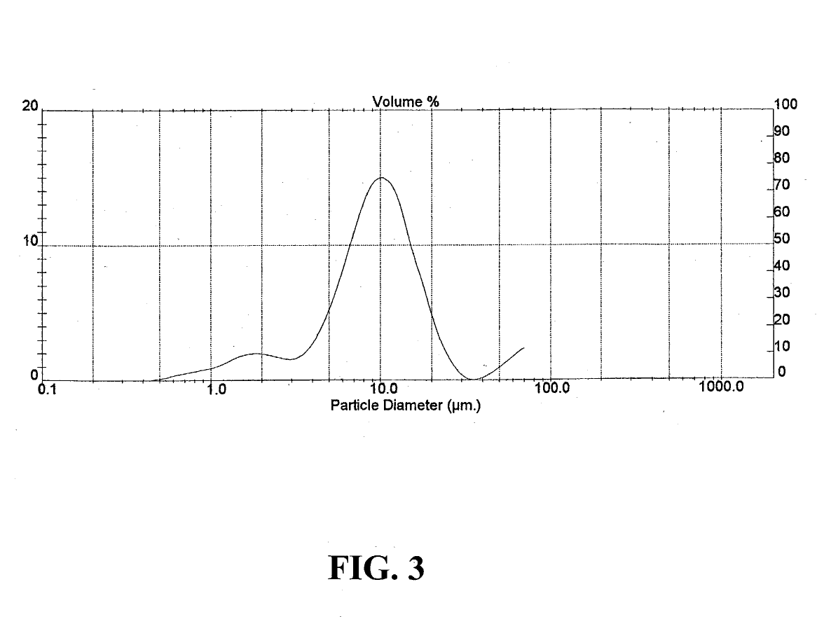

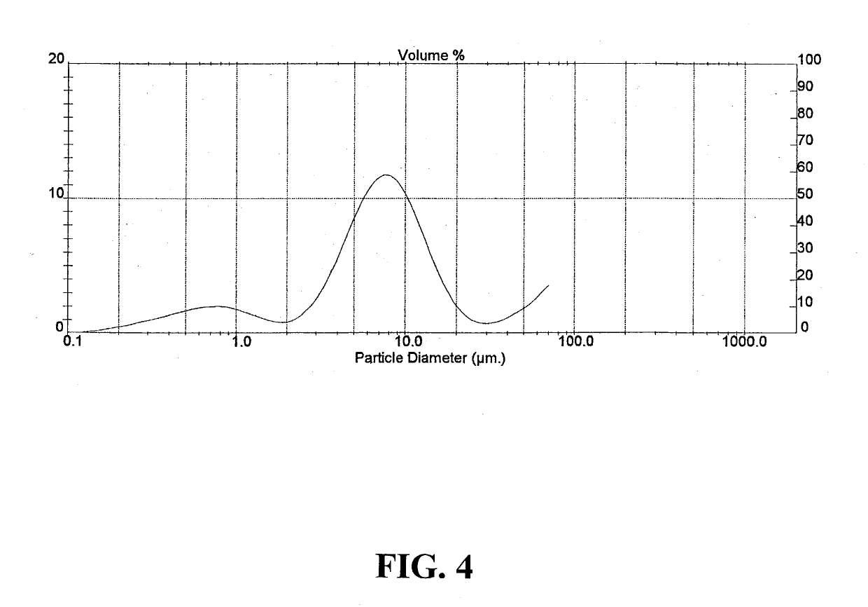

An examination of the results indicates that there was no appreciable change in particle size distribution over that time period, as shown in Table 2. Also, the formulation did not cake upon storage, and re-dispersed readily upon shaking. FIGS. 3 and 4 show the chromatograms of particle size distribution initially and after 4 weeks storage at 40° C.

TABLE 2

| | Sample (% based | | | |
|---|---|---|---|---|
| | on size of particles) | | | |
| | 10% | 50% | 90% | 99.5% |
| Initial (t = 0) | 0.83 μm | 5.88 μm | 29.5 μm | 75.28 μm |
| 1 week | 0.87 μm | 9.08 μm | 22.39 μm | 75.39 μm |
| 2 weeks | 0.81 μm | 8.68 μm | 18.65 μm | 74.45 μm |
| 4 weeks | 0.88 μm | 7.73 μm | 48.43 μm | 78.62 μm |

All references, including product descriptions, publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A sterile aqueous composition consisting essentially of:
triamcinolone acetonide,
water,
a tonicity-adjusting agent in an amount sufficient to provide the sterile aqueous composition with a physiologically-acceptable tonicity, and
a pH-adjusting agent in an amount sufficient to provide the aqueous composition with a pH of from about 5 to about 7.5.

2. The sterile aqueous composition according to claim 1, wherein the tonicity-adjusting agent is selected from the group consisting of sodium chloride, potassium chloride, dextrose, and mixtures thereof.

3. The sterile aqueous composition according to claim 2, wherein the tonicity-adjusting agent is sodium chloride.

4. The sterile aqueous composition according to claim 1, wherein the pH-adjusting agent is selected from the group consisting of buffering agents, inorganic bases, and mixtures thereof.

5. The sterile aqueous composition according to claim 2, wherein the pH-adjusting agent is selected from the group consisting of buffering agents, inorganic bases, and mixtures thereof.

7. The sterile aqueous composition according to claim 3, wherein the pH-adjusting agent is an inorganic base.

8. The sterile aqueous composition according to claim 1, wherein the triamcinolone acetonide is present at a concentration of about 1 mg to about 80 mg per 1 ml of water.

9. The sterile aqueous composition according to claim 8, wherein the triamcinolone acetonide is in the form of micronized particles, and wherein 95% of the particles have a particle size of no more than 100 microns.

10. The sterile aqueous composition according to claim 9, wherein at least 50% of the triamcinolone acetonide particles have a particle size of no more than 20 microns.

11. The sterile aqueous composition according to claim 10, wherein at least 50% of the triamcinolone acetonide particles have a particle size of no more than 20 microns, 90% have a particle size of no more than 50 microns, and 95% have a particle size of no more than 80 microns.

12. The sterile aqueous composition according to claim 19, wherein the triamcinolone acetonide is present in the sterile aqueous composition at from about 10 mg to about 40 mg per 1 ml of the composition.

13. The sterile aqueous composition according to claim 10, wherein the tonicity-adjusting agent is selected from the group consisting of sodium chloride, potassium chloride, dextrose, and mixtures thereof, the pH-adjusting agent is selected from the group consisting of buffering agents, inorganic bases, and mixtures thereof, and the triamcinolone acetonide is present at a concentration of about 10 mg to about 40 mg per 1 ml of the composition.

14. The sterile aqueous composition according to claim 1, wherein the composition is substantially free of at least one of a preservative, an alcohol, a suspending agent and a thickening agent.

15. The sterile aqueous composition according to claim 1, wherein the composition is substantially free at least two of a preservative, an alcohol, a suspending agent and a thickening agent.

16. The sterile aqueous composition according to claim 1, wherein the composition is substantially free of at least three of a preservative, an alcohol, a suspending agent and a thickening agent.

17. The sterile aqueous composition according to claim 17, wherein the composition is substantially free of a preservative, an alcohol, a suspending agent and a thickening agent.

18. The sterile aqueous composition according to claim 13, wherein the tonicity-adjusting agent is sodium chloride, the triamcinolone is present at a concentration of about 40 mg per 1 ml of the composition, and is substantially free of at least two of a preservative, an alcohol, a suspending agent and a thickening agent.

19. The sterile aqueous composition according to claim 13, wherein at least 50% of the triamcinolone acetonide particles have a particle size of no more than 20 microns, 90% have a particle size of no more than 50 microns, and 95% have a particle size of no more than 80 microns.

20. A sterile aqueous composition comprising triamcinolone acetonide, wherein the sterile aqueous composition has a pH of between about 6 and 7.5 and contains no more than 1 wt. % excipients other than tonicity and pH-adjusting agents.

21. The sterile aqueous composition according to claim 20, wherein the triamcinolone acetonide is in the form of micronized particles, and wherein 95% of the particles have a particle size of no more than 100 microns.

22. The sterile aqueous composition according to claim 21, wherein at least 50% of the triamcinolone acetonide particles have a particle size of no more than 20 microns.

23. The sterile aqueous composition according to claim 22, wherein at least 50% of the triamcinolone acetonide particles have a particle size of no more than 20 microns, 90% have a particle size of no more than 50 microns, and 95% have a particle size of no more than 80 microns.

24. The sterile aqueous composition according to claim 20, wherein the sterile aqueous composition contains no more than 0.5 wt. % excipients other than tonicity and pH-adjusting agents.

25. The sterile aqueous composition according to claim 20, wherein the sterile aqueous composition contains no more than 0.1 wt. % excipients other than tonicity and pH-adjusting agents.

26. The sterile aqueous composition according to claim 25, wherein the sterile aqueous composition is substantially free of excipients other than tonicity and pH-adjusting agents.

27. The sterile aqueous composition according to claim 23, wherein the triamcinolone is present in the sterile aqueous composition at from about 10 mg to about 40 mg per 1 ml of the composition, and the sterile aqueous composition is substantially free of excipients other than tonicity and pH-adjusting agents.

28. The sterile aqueous composition according to claim 20, wherein the sterile aqueous composition is a unit dose.

29. The sterile aqueous composition according to claim 21, wherein the sterile aqueous composition is a unit dose.

30. A sterile unit dose of triamcinolone acetonide comprising
a unit dose container within which resides a sterile aqueous composition consisting essentially of
triamcinolone acetonide,
water,
a tonicity-adjusting agent in an amount sufficient to provide the sterile aqueous composition with a physiologically-acceptable tonicity, and
a pH-adjusting agent in an amount sufficient to provide the aqueous composition with a pH of from about 5 to about 7.5.

31. The sterile aqueous composition according to claim 30, wherein the triamcinolone acetonide is in the form of micronized particles, and wherein 95% of the particles have a particle size of no more than 100 microns.

32. The sterile aqueous composition according to claim 31, wherein at least 50% of the triamcinolone acetonide particles have a particle size of no more than 20 microns.

33. The sterile aqueous composition according to claim 32, wherein at least 50% of the triamcinolone acetonide particles have a particle size of no more than 20 microns, 90% have a particle size of no more than 50 microns, and 95% have a particle size of no more than 80 microns.

34. The sterile aqueous composition according to claim 30, wherein the sterile aqueous composition contains no more than 0.5 wt. % excipients other than tonicity and pH-adjusting agents.

35. The sterile aqueous composition according to claim 30, wherein the sterile aqueous composition contains no more than 0.1 wt. % excipients other than tonicity and pH-adjusting agents.

36. The sterile aqueous composition according to claim 35, wherein the sterile aqueous composition is substantially free of excipients other than tonicity and pH-adjusting agents.

37. The sterile aqueous composition according to claim 30, wherein the tonicity-adjusting agent is selected from the group consisting of sodium chloride, potassium chloride, dextrose, and mixtures thereof, the pH-adjusting agent is selected from the group consisting of buffering agents, inorganic bases, and mixtures thereof, and the triamcinolone acetonide is present at a concentration of about 10 mg to about 40 mg per 1 ml of the composition.

38. The sterile aqueous composition according to claim 37, wherein at least 50% of the triamcinolone acetonide particles have a particle size of no more than 20 microns.

39. The sterile aqueous composition according to claim 38, wherein at least 50% of the triamcinolone acetonide particles have a particle size of no more than 20 microns, 90% have a particle size of no more than 50 microns, and 95% have a particle size of no more than 80 microns.

40. The sterile aqueous composition according to claim 39, wherein the sterile aqueous composition contains no more than 0.5 wt. % excipients other than tonicity and pH-adjusting agents.

41. The sterile aqueous composition according to claim 40, wherein the sterile aqueous composition contains no more than 0.1 wt. % excipients other than tonicity and pH-adjusting agents.

42. The sterile aqueous composition according to claim 41, wherein the sterile aqueous composition is substantially free of excipients other than tonicity and pH-adjusting agents.

43. A system for providing a unit dose of a sterile aqueous composition of triamcinolone acetonide comprising
a first container comprising a unit dose of triamcinolone acetonide sterilized by ethylene oxide, and
a second container comprising a sterile composition including water and a tonicity-adjusting agent.

44. The system according to claim 43, the second container further including a pH-adjusting agent.

45. The system according to claim 43, wherein the triamcinolone acetonide is present in the first container as a lyophilizate.

46. The system according to claim 44, wherein the first and second containers contain, in total, no more than 0.5 wt. % excipients other than tonicity and pH-adjusting agents.

47. The system according to claim 46, wherein the first and second containers contain, in total, no more than 0.1 wt. % excipients other than tonicity and pH-adjusting agents.

48. The system according to claim 47, wherein the first and second containers, in total, are substantially free of excipients other than tonicity and pH-adjusting agents.

49. The system according to claim 47, wherein the triamcinolone acetonide is in the form of micronized particles, and wherein 95% of the particles have a particle size of no more than 100 microns.

50. A process for preparing an aqueous sterile unit dose of triamcinolone acetonide comprising
(a) providing sterilized triamcinolone acetonide in a unit dose container,
(b) adding a sterile aqueous composition consisting essentially of water, a tonicity-adjusting agent in an amount sufficient to adjust the aqueous sterile unit dose with a physiologically-acceptable tonicity and, optionally, a pH-adjusting agent to adjust the pH of the aqueous composition to between 5 and 7.5, to the unit dose container to provide an aqueous sterile unit dose of triamcinolone acetonide,
wherein excipients other than the tonicity-adjusting agent and optional pH-adjusting agent in the sterile unit dose of triamcinolone acetonide are present at less than 1 wt. %.

51. The process according to claim 50, wherein the triamcinolone is sterilized after introduction into the unit dose container.

52. The process according to claim 51, wherein the sterilization is completed using ethylene oxide.

53. The sterile aqueous composition according to claim 51, wherein the triamcinolone acetonide is in the form of micronized particles, and wherein 95% of the particles have a particle size of no more than 100 microns.

54. The sterile aqueous composition according to claim 53, wherein at least 50% of the triamcinolone acetonide particles have a particle size of no more than 20 microns.

55. The sterile aqueous composition according to claim 54, wherein at least 50% of the triamcinolone acetonide particles have a particle size of no more than 20 microns, 90% have a particle size of no more than 50 microns, and 95% have a particle size of no more than 80 microns.

56. The process according to claim 50, wherein the sterile aqueous dose contains no more than 0.5 wt. % excipients other than tonicity and pH-adjusting agents.

57. The process according to claim 55, wherein the sterile aqueous dose contains no more than 0.1 wt. % excipients other than tonicity and pH-adjusting agents

58. The process according to claim 57, wherein the sterile aqueous dose is substantially free of excipients other than tonicity and pH-adjusting agents.

59. The process according to claim 57, wherein the tonicity-adjusting agent is sodium chloride and the pH-adjusting agent comprises an inorganic base.

60. The process according to claim 57, wherein the tonicity-adjusting agent is selected from the group consisting of sodium chloride, potassium chloride, dextrose, and mixtures thereof, the pH-adjusting agent is selected from the group consisting of buffering agents, inorganic bases, and mixtures thereof, and the triamcinolone acetonide is present at a concentration of about 10 mg to about 40 mg per 1 ml of the composition.

61. A process for preparing a sterile aqueous composition of triamcinolone acetonide comprising
(a) lyophilizing an aqueous triamcinolone acetonide composition, and
(b) reconstituting the lyophilized unit dose of triamcinolone acetonide with a sterile aqueous composition comprising water, a tonicity-adjusting agent and, optionally, a pH-adjusting agent to provide an aqueous sterile unit dose of triamcinolone acetonide.

62. The process according to claim 61, wherein the excipients other than the tonicity-adjusting agent and optional pH-adjusting agent are present in the sterile reconstituted aqueous composition at less than 1 wt. %.

63. The process according to claim 62, wherein the sterile aqueous composition used to reconstitute the lyophilizate further comprises a pH-adjusting agent in an amount sufficient to adjust the pH of the reconstituted sterile aqueous composition to between 5 and 7.5.

64. The process according to claim 62, wherein the excipients other than the tonicity-adjusting agent and optional pH-adjusting agent are present in the sterile reconstituted aqueous composition at less than 0.5 wt. %.

65. The process according to claim 63, wherein the excipients other than the tonicity-adjusting agent and optional pH-adjusting agent are present in the sterile reconstituted aqueous composition at less than 0.1 wt. %.

66. The process according to claim 61, wherein the lyophilized triamcinolone acetonide is sterilized by ethylene oxide.

67. The process according to claim 66, wherein the triamcinolone acetonide composition is lyophilized in a unit dose container.

68. A method for treating macular edema comprising administering a sterile aqueous triamcinolone acetonide composition by intravitreal injection, the aqueous composition comprising
triamcinolone acetonide,
water,
a tonicity-adjusting agent in an amount sufficient to provide the sterile aqueous composition with a physiologically-acceptable tonicity, and
a pH-adjusting agent in an amount sufficient to provide the aqueous composition with a pH of from about 5 to about 7.5.

69. The sterile aqueous composition according to claim 68, wherein the tonicity-adjusting agent is selected from the group consisting of sodium chloride, potassium chloride, dextrose, and mixtures thereof.

70. The sterile aqueous composition according to claim 69, wherein the tonicity-adjusting agent is sodium chloride.

71. The sterile aqueous composition according to claim 68, wherein the pH-adjusting agent is selected from the group consisting of buffering agents, inorganic bases, and mixtures thereof.

72. The sterile aqueous composition according to claim 69, wherein the pH-adjusting agent is selected from the group consisting of buffering agents, inorganic bases, and mixtures thereof.

73. The sterile aqueous composition according to claim 70, wherein the pH-adjusting agent is an inorganic base.

74. The sterile aqueous composition according to claim 69, wherein the triamcinolone acetonide is present at a concentration of about 3 mg to about 50 mg per 1 ml of the composition.

75. The sterile aqueous composition according to claim 74, wherein the triamcinolone acetonide is in the form of micronized particles, and wherein 95% of the particles have a particle size of no more than 100 microns.

76. The sterile aqueous composition according to claim 75, wherein at least 50% of the triamcinolone acetonide particles have a particle size of no more than 20 microns.

77. The sterile aqueous composition according to claim 76, wherein at least 50% of the triamcinolone acetonide particles have a particle size of no more than 20 microns, 90% have a particle size of no more than 50 microns, and 95% have a particle size of no more than 80 microns.

78. The sterile aqueous composition according to claim 76, wherein the tonicity-adjusting agent is selected from the group consisting of sodium chloride, potassium chloride, dextrose, and mixtures thereof, and the pH-adjusting agent is selected from the group consisting of buffering agents, inorganic bases, and mixtures thereof.

79. The sterile aqueous composition according to claim 78, wherein the triamcinolone acetonide is present at a concentration of about 10 mg to about 40 mg per 1 ml of the composition.

80. The sterile aqueous composition according to claim 77, wherein the tonicity-adjusting agent is selected from the group consisting of sodium chloride, potassium chloride, dextrose, and mixtures thereof, the pH-adjusting agent is selected from the group consisting of buffering agents, inorganic bases, and mixtures thereof, and the triamcinolone acetonide is present at a concentration of about 10 mg to about 40 mg per 1 ml of the composition.

81. The sterile aqueous composition according to claim 68, wherein the composition is substantially free of at least one of a preservative, an alcohol, a suspending agent and a thickening agent.

82. The method according to claim 68, further comprising diagnosing the ocular edema by angiography.

83. The method according to claim 80, wherein the angiography is completed using a tricarbocyanine dye.

84. The method according to claim 81, wherein the tricarbocyanine dye is indocyanine dye.

85. The method according to claim 80, further comprising completing photodynamic therapy.

86. The method according to claim 85, wherein the photodynamic therapy is completed using a porphyrin.

87. The method according to claim 68, further comprising the administration of an anti-vascular endothelial growth factor.

88. A method for treating a patient having a health-related condition comprising administering to a patient afflicted with such disease a therapeutically-effective amount of a sterile aqueous composition comprising micronized triamcinolone acetonide, wherein the sterile aqueous composition has a pH of between about 5 and 7.5 and contains no more than 1 wt. % excipients other than tonicity and pH-adjusting agents, wherein the health-related condition is selected from the group consisting of ophthalmic disease, endocrine disorder, rheumatic disorder, edematous condition, pain, allergic condition, inflammatory condition, collagen disease, gastrointestinal disease, neoplastic disease, dermatologic disease, respiratory disease, hematologic disease, temporal arteritis and myasthenia gravis.

89. A method for treating a patient having diabetic macular edema comprising administering to a patient afflicted with such edema a therapeutically-effective amount of a sterile aqueous composition comprising micronized triamcinolone acetonide, wherein the sterile aqueous composition has a pH of between about 5 and 7.5 and contains no more than 1 wt. % excipients other than tonicity and pH-adjusting agents.

89. A method for treating a patient having a CNV comprising administering to a patient afflicted with the CNV a therapeutically-effective amount of a sterile aqueous composition comprising micronized triamcinolone acetonide, wherein the sterile aqueous composition has a pH of between about 5 and 7.5 and contains no more than 1 wt. % excipients other than tonicity and pH-adjusting agents.

90. The method according to claim 89, further comprising treating the CNV with laser therapy.

91. The method according to claim 90, wherein the laser therapy is photodynamic therapy.

92. The method according to claim 90, wherein the laser therapy is dye-enhanced photocoagulation.

94. A method for treating a patient afflicted with edema or bleeding following vitreal or retinal surgery comprising administering via injection to the patient a therapeutically-effective amount of a sterile aqueous composition comprising micronized triamcinolone acetonide, wherein the sterile aqueous composition has a pH of between about 5 and 7.5 and contains no more than 1 wt. % excipients other than tonicity and pH-adjusting agents.
